SEQUENCE LISTING (41 sequences)

SEQ ID NO: 1 - protein, 45 aa, Homo sapiens
PTAQSSGSLYGSQIYALCNQFYTPAATGLYVDQYLYHYCVVDPQE

SEQ ID NO: 2 - protein, 45 aa, Mus musculus
PTAQASGSMYGSQIYMICNQFYTPGGTGLYVDQYLYHYCVVDPQE

SEQ ID NO: 3 - protein, 45 aa, Rattus norvegicus
PTAQASGSMYGSQIYTICSQFYTPGGTGLYVDQYLYHYCVVDPQE

SEQ ID NO: 4 - protein, 41 aa, Gallus gallus
PQAQMSSGYYYSPLLAMCSQAYGSTYLNQYIYHYCTVDPQE

SEQ ID NO: 5 - protein, 45 aa, Canis familiaris
PTAQASGSLYSSQIYAMCNQFYASTATGLYMDQYLYHYCVVDPQE

SEQ ID NO: 6 - protein, 12 aa, Rattus norvegicus
TICSQFYTPGGT

SEQ ID NO: 7 - protein, 13 aa, Rattus norvegicus
YTICSQFYTPGGT

SEQ ID NO: 8 - protein, 13 aa, Rattus norvegicus
TICSQFYTPGGTG

SEQ ID NO: 9 - protein, 14 aa, Rattus norvegicus
YTICSQFYTPGGTG

SEQ ID NO: 10 - protein, 14 aa, Rattus norvegicus
IYTICSQFYTPGGT

SEQ ID NO: 11 - protein, 14 aa, Rattus norvegicus
TICSQFYTPGGTGL

SEQ ID NO: 12 - protein, 15 aa, Rattus norvegicus
IYTICSQFYTPGGTG

SEQ ID NO: 13 - protein, 15 aa, Rattus norvegicus
YTICSQFYTPGGTGL

SEQ ID NO: 14 - protein, 15 aa, Rattus norvegicus
QIYTICSQFYTPGGT

SEQ ID NO: 15 - protein, 16 aa, Rattus norvegicus
QIYTICSQFYTPGGTG

SEQ ID NO: 16 - protein, 16 aa, Rattus norvegicus
YTICSQFYTPGGTGLY

SEQ ID NO: 17 - protein, 17 aa, Rattus norvegicus
YTICSQFYTPGGTGLYV

SEQ ID NO: 18 - protein, 18 aa, Rattus norvegicus
YTICSQFYTPGGTGLYVD

SEQ ID NO: 19 - protein, 19 aa, Rattus norvegicus
IYTICSQFYTPGGTGLYVD

SEQ ID NO: 20 - protein, 20 aa, Rattus norvegicus
QIYTICSQFYTPGGTGLYVD

SEQ ID NO: 21 - protein, 21 aa, Rattus norvegicus
SQIYTICSQFYTPGGTGLYVD

SEQ ID NO: 22 - protein, 15 aa, Rattus norvegicus
TICSQFYTPGGTGLY

SEQ ID NO: 23 - protein, 16 aa, Rattus norvegicus
TICSQFYTPGGTGLYV

SEQ ID NO: 24 - protein, 17 aa, Rattus norvegicus
TICSQFYTPGGTGLYVD

SEQ ID NO: 25 - protein, 21 aa, Rattus norvegicus
GSQIYTICSQFYTPGGTGLYV

SEQ ID NO: 26 - protein, 20 aa, Rattus norvegicus
GSQIYTICSQFYTPGGTGLY

SEQ ID NO: 27 - protein, 19 aa, Rattus norvegicus
GSQIYTICSQFYTPGGTGL

SEQ ID NO: 28 - protein, 18 aa, Rattus norvegicus
GSQIYTICSQFYTPGGTG

SEQ ID NO: 29 - protein, 9 aa, Artificial Sequence
  Description of Artificial Sequence Illustrative Tat peptide
RKKRRQRRR

SEQ ID NO: 30 - DNA, 19 bp, Artificial Sequence
  Description of Artificial Sequence Primer
ctgtctatgctcgtcatcg

SEQ ID NO: 31 - DNA, 20 bp, Artificial Sequence
  Description of Artificial Sequence Primer
cattcccgatctaatgacgc

SEQ ID NO: 32 - DNA, 24 bp, Artificial Sequence
  Description of Artificial Sequence Primer
tccgctgcagtccgttcaagtctt

SEQ ID NO: 33 - DNA, 24 bp, Artificial Sequence
  Description of Artificial Sequence Primer
gccaaacttcttggattcgcagcg

SEQ ID NO: 34 - protein, 22 aa, Rattus norvegicus
GSQIYTICSQFYTPGGTGLYVD

SEQ ID NO: 35 - protein, 22 aa, Rattus norvegicus
TKVNERYELCDTYPALLAVPAN

SEQ ID NO: 36 - protein, 120 aa, Rattus norvegicus
MDCYRRYAAVILVMLSMVLHILHSLPDGDLIIQGCPECKLKENKYFSKLGAPIYQCMGCCFSRAYPTPARSKKTMLVPKNITSQATCCVAKSFTKATVMGNARVENHTDCHCSTCYYHKS

SEQ ID NO: 37 - protein, 120 aa, Rattus norvegicus
MDCYRRYAAVILVMLSMVLHILHSLPDGDLIIQGCPECKLKENKYFSKLGAPIYQCMGCCFSRAYPTPARSKKTMLVPKDITSQATCCVAKSFTKATVMGNARVEDHTDCHCSTCYYHKS

SEQ ID NO: 38 - protein, 120 aa, Rattus norvegicus
MDCYRRYAAVILVMLSMVLHILHSLPDGDLIIQGCPECKLKENKYFSKLGAPIYQCMGCCFSRAYPTPARSKKKKKVPKNITSQATCCVAKSFTKATVAAAARVENHTDCHCSTCYYHKS

SEQ ID NO: 39 - protein, 130 aa, Rattus norvegicus
MMKSIQLCILLWCLRAVCCHSCELTNITISVEKEECRFCISINTTWCEGYCYTRDLVYKDPARPNTQKVCTFKELVYETIRLPGCARHSDSLYTYPVATECHCGKCDSDSTDCTVRGLGPSYCSFGEMKE

SEQ ID NO: 40 - protein, 127 aa, Rattus norvegicus
MMKSIQLCILLWCLRAVCCHSCELTNITISVEKEECRFCISTWCEGYCYTRDLVYKDPARPNTQKVCTFKELVYETIRLPGCARHSDSLYTYPVATECHCGKCDSDSTDCTVRGLGPSYCSFGEMKE

SEQ ID NO: 41 - protein, 12 aa, Homo sapiens
ALCNQFYTPAAT

(19) United States
(12) Patent Application Publication
(10) Pub. No.: US 2004/0242476 A1
(43) Pub. Date: Dec. 2, 2004

(54) Male contraceptives

(75) Inventor: C Yan Cheng, Staten Island, NY (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: The Population Council, Inc, New York, NY (US)

(21) Appl. No.: 10/481,279

(22) PCT Filed: Jun. 19, 2002
(86) PCT No.: PCT/US02/19413

Related U.S. Application Data
(60) Provisional application No. 60/299,313, filed on Jun. 19, 2001.

(51) Int. Cl. A61K 38/22

(52) U.S. Cl. 514/12

(57) ABSTRACT

Disclosed are male contraceptives containing a peptide and analogs thereof having an amino acid sequence corresponding to the second extracellular domain of a mammalian occludin. The peptides may be linked to a carrier targeting testicular cells, preferably to a modified follicle stimulating hormone. Methods of producing such a contraceptive, and a method of use are also disclosed.

### CROSS-REFERENCE TO RELATED APPLICATIONS

The application claims the benefit of U.S. Provisional Application No. 60/299,313, filed on Jun. 19, 2001, the disclosure of which is incorporated herein by reference.

### GOVERNMENT SUPPORT

The invention described herein was supported in part by National Institutes of Health grant No. U54-HD-13541-20S. Therefore, the government may have rights in the invention.

### BACKGROUND OF THE INVENTION

Female reproductive systems have been studied extensively over the years. Numerous methods of female contraception have been developed. In contrast, the physiology of the male reproductive system is not as well understood. Consequently, few options for contraception are currently available for men.

For the past several decades, development of new male contraceptives has largely been focused on manipulating the hypothalamus-pituitary-testicular axis to disrupt spermatogenesis (See Paulsen, et al., 1994). Administration of either high doses of testosterone or synthetic progestins can inhibit pituitary gonadotropin secretion which in turn leads to oligospermia or azoospermia (Frick, et al. 1977). Although this inhibitory effect on spermatogenesis is reversible, the exogenous administration of steroids or polypeptide hormones tends to interfere with the hormonal balance and induces undesirable side effects. When the androgen level is affected, other secondary sexual characteristics are affected. Additionally, androgen, a male sex hormone, has diversified effects in the human body. Some of the androgen regulated physiological end-points are not yet known, as such, the potential side-effects may not be known until years later.

Throughout spermatogenesis, a series of molecular, biochemical, and cellular events take place. These events lead to the production of four spermatids from a single spermatogonium via two consecutive reductive divisions during meiosis (for review, see de Kretser and Kerr, 1988; Byers et al., 1993). On the other hand, developing germ cells must also translocate progressively from the basal to the adluminal compartment of the seminiferous epithelium so that fully developed spermatids (spermatozoa) can be released into the tubular lumen during spermiation. In addition, the inter-Sertoli tight junctions (TJs) that constitute the blood-testis barrier (BTB) must be disrupted and reassembled periodically to allow the timely passage of preleptotene spermatocytes across the BTB, entering into the adluminal compartment to continue their development. This timely movement of developing germ cells across the BTB and the epithelium is essential to the completion of spermatogenesis.

The inter-Sertoli TJs that create the blood-testis barrier play an important role in the testis. First, they serve as a fence between the seminiferous epithelium and the basal lamina restricting paracellular transport of molecules. Second, they constitute the major part of the blood-testis barrier that segregates germ cell development from the systemic circulation creating a favorable milieu for spermatogenesis (Dym et al., 1970). Third, TJs create and maintain cell polarity. Several TJ-associated proteins such as ZO-1 (Byers et al., 1991; Pelletier et al., 1997), cingulin (Byers et al., 1993), occludin (Moroi et al., 1998) and claudin-1, -3, -5, -7, -8, and -11 (for review, see Fanning et al., 1999; Tsukita and Furuse, 2000) have been found in the testis. Among these proteins, only ZO-1, a cytoplasmic protein, has been extensively studied in the testis (Byers et al., 1991; Pelletier et al., 1997; Chung et al., 1999; Wong et al., 2000).

Occludin is a 65 kDa integral membrane protein localized at TJ strands (Furuse et al., 1993; Fujimoto et al., 1995; Saitou et al., 1997). Occludin consists of four transmembrane domains, a long carboxy-terminal cytoplasmic domain, a short amino-terminal cytoplasmic domain, two extracellular loops and one intracellular loop. Among these domains, the first extracellular domain is rich in Tyr and Gly content (approximately 60%) (Ando-Akatsuka et al., 1996). These characteristics are well conserved among the mammalian species (Ando-Akatsuka et al., 1996).

Induction of occludin expression is detected at the time when TJs are being assembled as assessed by transepithelial electrical resistance (TER). This demonstrates that occludin is required for TJ formation. Both ZO-1 and occludin expression are induced at the time when inter-Sertoli TJs are being assembled, consistent with the observation that the cytoplasmic domain of occludin is associated with ZO-1 at a 1:1 molar ratio (Furuse et al., 1994). These results are also consistent with the belief that ZO-1 acts as a linker to bridge an integral membrane TJ protein such as occludin, and the actin-based cytoskeleton during TJ biogenesis (Furuse et al., 1994; Fanning et al., 1998). The timely induction of occludin expression during TJ assembly is further consistent with several reports that an elevated level of phosphorylated occludin was observed during TJ assembly (Sakakibara et al., 1997; Wong et al., 1997b).

Functional analyses of occludin in different epithelial systems have shown that occludin plays a crucial role in the assembly of TJs (for review, see Matter and Balda, 1999; Mitic and Anderson, 1998; Tsukita and Furuse, 1999). For instance, an increase in TER was detected in MDCK cells following transfection with a full-length occludin cDNA (Balda et al., 1996; McCarthy et al., 1996). However, it has also been found that occludin-deficient embryonic stem cells are capable of differentiating into polarized epithelial cells bearing TJs (Saitou et al., 1998). This result suggests that claudin (another TJ-integral transmembrane protein of which at least 18 different species have been found) or other yet-to-be identified TJ-integral proteins could supersede the role of occludin in TJ assembly, which may also associate with the underlying cytoplasmic TJ-protein, ZO-1. Other studies have shown that claudin-1, -3, -5, -7, -8, and -11 are present in the testis, suggesting that epithelial cells utilize other TJ proteins to construct TJs (for review, see Mitic et al., 2000).

The effects of synthetic occludin peptides have been investigated in vitro. For example, one study used synthetic peptides corresponding to the first external loop of occludin to examine its role in cell-cell adhesion. When occludin-transfected fibroblasts were incubated with peptide corresponding to the first external loop of occludin, occludin-induced cell adhesion was inhibited. (Van Itlalie and Anderson, 1997). This observation prompted speculation that the first external loop is responsible for cell-cell adhesion. In another study, the addition of a peptide homologous to the first external loop of chick occludin to Xenopus A6 cell cultures prevented the resealing of the TJs. In contrast, a 10-amino acid peptide corresponding to the second extracellular domain had no effect (Lacaz-Viera et al., 1999). However, Wong and Gumbiner (1997) have demonstrated that a 44-amino acid peptide synthesized based on the entire first external loop of chick occludin failed to disrupt TJs in cultured Xenopus kidney epitheilial (A6) cells, whearas a 44-amino acid peptide covering the entire second external loop did perturb TJ assembly in A6 cells.

### SUMMARY OF THE INVENTION

One aspect of the present invention is directed to a male contraceptive containing a peptide having an amino acid sequence corresponding to the second extracellular domain of a mammalian occludin, or an analog of the peptide, and a carrier. The peptide or analog thereof disrupts inter-Sertoli cell tight junctions in vivo. In some embodiments, the analog corresponds to a fragment of the second extracellular domain of a mammalian occludin. The sequence of the fragment might differ from the native corresponding fragment in terms of one or more amino acid substitutions, additions and/or deletions.

The contraceptives may be administered orally or parenterally (e.g., by way of intratesticular injection). In some embodiments, the peptide is linked to a ligand (thus forming a conjugate) that specifically targets testicular cells. In other preferred embodiments, the ligand is a peptide and is linked to the occludin peptide via a peptide bond, such that the occludin peptide and the ligand are linked in the form of a fusion protein. In more preferred embodiments, the ligand contains at least a portion of the binding domain of follicle stimulating hormone (FSH). Nucleic acids encoding the occludin peptides and the fusion proteins, and constructs (e.g., vectors and hosts) containing same, are also provided. Methods of making the contraceptives and using the peptides or the nucleic acids to achieve a contraceptive effect in a mammal such as a human, are further provided.

Under normal circumstances, the blood-testis barrier formed by the inter-Sertoli cell tight junctions defines a protective environment called the seminiferous epithelium that allows germ cells to develop and mature into fully functional sperm cells. Without being bound by any particular theory of operation, Applicant believes that by disrupting the tight junctions formed by and between Sertoli cells, the peptides of the present invention also cause disruption of the blood-testis barrier (BTB), leading to the influx of the body's immune cells into the seminiferous epithelium. The immune cells recognize the germ cells as “foreign” to the body and destroy them. Consequently, sperm cell count is lowered to an infertile level.

### BRIEF DESCRIPTION OF THE DRAWINGS

FIG. 1 is a graph illustrating the assembly of inter-Sertoli tight junction (TJ)-permeability barrier in vitro.

FIGS. 2A and 2C are gels and 2B and 2D are graphs illustrating changes of the steady-state occludin mRNA level during the assembly of inter-Sertoli TJs in vitro.

FIGS. 3A and B are chromatograms illustrating purification of the occludin peptide by HPLC and microsequencing for identity confirmation.

FIGS. 4A and B are graphs illustrating the effect of a 22-amino acid occludin peptide corresponding to the second extracellular loop of rat occludin (residues 209-230) on the inter-Sertoli TJ permeability barrier in vitro.

FIG. 5 is a graph illustrating changes in testicular weight after intratesticular injection of an occludin peptide.

FIGS. 6A-L are photographs depicting a microscopic view of the antispermatogenic effects of the 22-amino acid synthetic occludin peptide following its administration to adult rats via intratesticular injection.

FIGS. 7A and B are graphs illustrating reversible disruption of the blood-testis barrier (BTB) following an intratesticular injection of the synthetic occludin or myotubularin peptide.

FIG. 8 is a schematic representation of the orientation of human occludin relative to the cell membrane.

### DETAILED DESCRIPTION

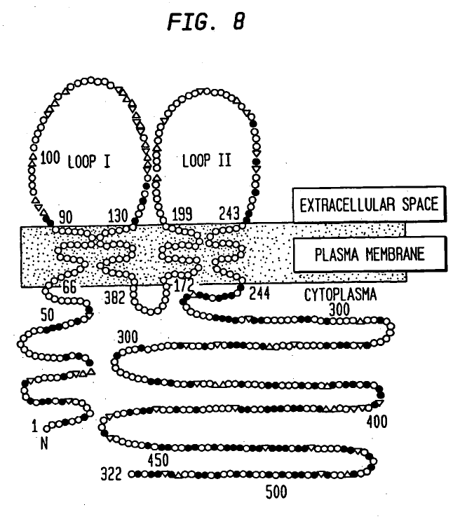

The peptides of the present invention (also termed “occludin peptides”) correspond to the second extracellular loop of a mammalian occludin, or an analog thereof, that disrupts tight junctions between Sertoli cells. A schematic representation of human occludin is illustrated in FIG. 8. As can be seen, the human occludin peptide contains first and second intracellular loops/domains. An alignment of the sequences representing the second extracellular loop of various mammalian occludins is set forth in Table 1. The alignment shows that mammalian occludins possess substantial homology or sequence similarity in this domain.

TABLE 1

| | | |
|---|---|---|
| Human | PTAQ-SSGSLYGSQIYALCNQFYTPAATGLYVDQYLYHYCVVDPQE-COOH | (199-243) |
| Mouse | PTAQ-ASGSMYGSQIYMICNQFYTPGGTGLYVDQYLYHYCVVDPQE-COOH | (197-241) |
| Rat | PTAQ-ASGSMYGSQIYTICSQFYTPGGTGLYVDQYLYHYCVVDPQE-COOH | (199-243) |
| Chicken | PQAQM-SSGYYYSPLLAMCSQ---AYGST-YLNQYIYHYCTVDPQE-COOH | (187-227) |
| Dog | PTAQ-ASGSLYSSQIYAMCNQFYASTATGLYMDQYLYHYCVVDPQE-COOH | (198-242) |

See, Mitic et al., Ann. Rev. Physiol. 60:121-142 (1998), and Genbank Accession Numbers NP-112619 (rat occludin), NP-032782 (mouse occludin), AAC50451 (human occludin) and A49467 (chicken occludin). In addition, rat kangaroo shows significant homology in this region. The second extracellular loops in occludins of yet other mammalian species can be identified in accordance with standard techniques, such as by probing genomic libraries with oligonucleotides that hybridize to the second extracellular loop or a fragment thereof. To facilitate interpretation of the sequence information, Table 2 sets forth amino acid names and both 3- and 1-letter symbols.

TABLE 2
Amino acid symbols

| Amino Acid | Three-letter symbol | One-letter symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylanine | Phe | F |
| Proline | Pro | P |
| Serine | Ser | S |
| Theronine | Tbr | T |
| Tryptophan | Trp | W |
| Tyrosine | Tyr | Y |
| Valine | Val | V |

In general, analogs of the occludin peptide are full-length versions (of the sequences set forth in Table 1), or fragments thereof, which may have one or more naturally occurring or synthetic (e.g. modified) amino acid substitutions, additions and/or deletions, provided that the analog disrupts tight junction formation between Sertoli cells in vivo. In the case where the analog contains one or more amino acid substitutions, conservative changes are preferred. For example, the hydrophilic amino acids such as S, Q, G and T may be replaced with another hydrophilic amino acid residue such as D, N or G. Likewise, a hydrophobic amino acid residue such as Y may be replaced by another hydrophobic residue, e.g., R, I, K or L. Hydrophobic substitutions can be advantageous from the standpoint of solubility of the peptide in a given carrier. Non-conservative substitutions are also permissible, provided that the analog retains ability to disrupt tight junction formation.

Preferred analogs are fragments that correspond to the second extracellular loop. In general, fragments that disrupt tight junction formation contain from about 12 to all (e.g., from 43-45) amino acid residues corresponding to the second extracellular loop of a mammalian occludin, e.g., peptides having 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 amino acid residues. Preferred fragments are those that are most external or outermost to the cell membrane (see FIG. 8), and correspond to the portion of the loop that participates in formation of the interlocking domain of the tight junction. Referring again to the rat occludin sequence set forth in Table 1, preferred analogs contain the 12-amino acid sequence TICSQFYTPGGT (corresponding to amino acid residues 214-225). In humans, the corresponding 12-amino acid sequence is ALCNQFYTPAAT (also corresponding to amino acid residues 214-255). Thus, in addition to this 12-amino acid peptide per se, other analogs containing this peptide may be deduced simply by adding one or more of the naturally occurring amino acid residues that flank the sequence at either terminus. Referring again to table 1, representative analogs containing this 12-amino acid sequence include the following:

NH₂-TICSQFYTPGGT-COOH
NH₂-YTICSQFYTPGGT-COOH
NH₂-TICSQFYTPGGTG-COOH
NH₂-YTICSQFYTPGGTG-COOH
NH₂-IYTICSQFYTPGGT-COOH
NH₂-TICSQFYTPGGTGL-COOH
NH₂-IYTICSQFYTPGGTG-COOH
NH₂-YTICSQFYTPGGTGL-COOH
NH₂-QIYTICSQFYTPGGT-COOH
NH₂-QIYTICSQFYTPGGTG-COOH
NH₂-YTICSQFYTPGGTGLY-COOH
NH₂-YTICSQFYTPGGTGLYV-COOH
NH₂-YTICSQFYTPGGTGLYVD-COOH
NH₂-IYTICSQFYTPGGTGLYVD-COOH
NH₂-QIYTICSQFYTPGGTGLYVD-COOH
NH₂-SQIYTICSQFYTPGGTGLYVD-COOH
NH₂-TICSQFYTPGGTGLY-COOH
NH₂-TICSQFYTPGGTGLYV-COOH
NH₂-TICSQFYTPGGTGLYVD-COOH
NH₂-GSQIYTICSQFYTPGGTGLYV-COOH
NH₂-GSQIYTICSQFYTPGGTGLY-COOH
NH₂-GSQIYTICSQFYTPGGTGL-COOH
NH₂-GSQIYTICSQFYTPGGTG-COOH

Described in terms of the numbering (e.g., TICSGFYTPGGT denoted as (214-225), some other analogs containing this sequence include amino acid residues (213-225), (212-225), (211-225), (210-225), (209-225), (208-225), (207-225), (206-225), (205-225), (204-225), (203-225), (202-225), (201-225), (200-225), (199-225), (214-226), (214-227), (214-228), (214-229), (214-230), (214-231), (214-232), (214-233), (214-234), (214-235), (214-236), (214-237), (214-238), (214-239), (214-240), (214-241), (214-242), (214-243), (213-226), (212-227), (211-228), (210-229) and (209-230). Using the same scheme, lists of suitable analogs derived from other mammalian occludins, including humans, can be easily generated.

Other analogs can be tested to determine whether they disrupt inter-Sertoli cell tight junctions in the testis using standard procedures described in the literature. See, Lui WY, Lee WM and Cheng CY (2001) Transforming growth factor-b3 perturbs the inter-Sertoli tight junction permeability barrier in vitro possibly mediated via its effects on occludin, zonula occludins-1, and claudin-11, Endocrinology 142:1865-1877; Chung NPY and Cheng CY (2001) Is cadmium chloride-induced inter-Sertoli tight junction permeability barrier disruption a suitable in vitro model to study the events of junction disassembly during spermatogenesis in the rat testis?, Endocrinology 142:1878-1888; and Grima J, Wong CSC, Zhu LJ, Zong SD and Cheng CY (1998) Testin secreted by Sertoli cells is associated with the cell surface and its expression correlates with the disruption of Sertoli-germ cell junctions but not the inter-Sertoli tight junction., J. Biol. Chem. 273:21040-21053. A monolayer suspension of Sertoli cells is prepared (e.g., where Sertoli cells are cultured on Matrigel-coated bicameral units at a density of approximately 1×10₆ cells/cm²). Then, a pulse of current (approximately 20 μA) is applied across the Sertoli cell epithelium between two electrodes (e.g., silver-silver chloride) for a short time (e.g., about 2 seconds), and the resistance is quantified (e.g., using a Millicell electrical resistance system (Millipore Corp)). The resistance is multiplied by the surface area of the filter (approximately 0.6 cm²) to yield the resistance in ohms x cm². The analog is then added to the suspension. If the analog perturbs the tight junction barrier, the electricial resistance across the Sertoli cell epithelium will be reduced (e.g., from about 100 ohms x cm² to about 40-80 ohms x cm² or even less).

The peptides and analogs of the present invention may be prepared in accordance with standard techniques such as solid phase peptide synthesis or via genetic engineering such as by expression of DNAs encoding the peptides in recombinant hosts (e.g., cells). Known suitable hosts may include bacterial, yeast, fungal, mammalian, and plant cells. Parameters such as codon preference, choice of regulatory sequences, and hosts etc., may be optimized in accordance with known procedures.

The peptides of the present invention are useful as a male contraceptive. Thus, they may be formulated for administration to any male mammal, especially a human. The amount of peptide administered will depend upon several factors such as the weight and overall health of the male and the manner of administration. In general, however, dosage will be in the range of approximately 0.1-10 mg/testis. The male will ordinarily become infertile within approximately 2-4 weeks from the time of administration. The effects of the peptides are temporary. The male will remain infertile for approximately 6 weeks until the effects wear off and fertility is re-established. These time periods will vary from individual to individual.

Suitable modes of administration of the contraceptive include oral and parenteral administration such as by way of subcutaneous or intratesticular injection. In addition, delivery of the peptide to the testis may be targeted such as by coupling the peptide with a ligand e.g., follicle-stimulating hormone (FSH), or a derivative thereof, that has diminished (compared to the wild type protein) or, preferably, completely lacks hormonal activity, that selectively binds testicular cells. The ligand may be peptidic or non-peptidic in nature. Thus, the ligand may be coupled to the occludin peptide to form a conjugate in accordance with standard techniques and reagents, such as by way of chemical cross-linking. In embodiments where the ligand is a peptide, the occludin peptide and the ligand may be linked via a peptide bond so as to produce a fusion protein. The ligand is chosen to target receptors present in greater abundance or exclusively on or in testicular cells, and preferably Sertoli cells. Thus, the conjugates or fusion proteins will preferentially or selectively bind such cells relative to cells that express the receptor in lesser quantities or not at all.

FSH is a heterodimeric glycoprotein consisting of two non-covalently linked α and β subunits (for review, see Pierce et al., 1981). FSH binds to the FSH receptor present on Sertoli cells (for review, see Fritz et al., 1978) via its receptor binding sites that reside within amino acid residues 93-99 in the C-terminal region of the β subunit (Lindau Shepard et al., 1994), and His90 and Lys91 of the α subunit (Zeng et al., 1995). FSH receptors are not known to be present on other cell types. Amino acid residues Asn52 and Asn78 on the α subunit, and residues Asn7 and Asn24 on the β subunit, confer hormonal activity (for review, see Rose et al., 2000). It has been reported that deglycosylation of the two Asn residues of the α-subunit results in a significant decrease in biopotency to about 41% of the wild type (Bishop et al., 1994). Thus, site-directed mutagenesis at these four sites will yield a FSH molecule having little or negligible (functionally insignificant) hormonal activity while maintaining binding affinity for Sertoli cells. Such a mutated FSH coding sequence can then be inserted into an expression vector and transfected into a host (e.g., CHO cells) for the purposes of producing a recombinant protein (FSH fused to the peptide) or a conjugate wherein the FSH is linked to the peptide (e.g., by a chemical cross-linking agent) (Keene et al., 1989; Van Wezenbeek et al., 1990). In this case, subcutaneous (e.g., intramuscular) administration would be particularly suitable. The choice of other suitable ligands, and modification thereof to maintain requisite binding affinity, but wherein other native functions are effectively eliminated, are within the level of skill in the art of cell-specific delivery systems.

Likewise, the choice of carrier may be made and optimized for any given mode of administration, and is within the level of skill in the pharmaceutical arts. Selection of other ingredients that may be useful in formulating the peptide for any given mode of administration, such as adjuvants, diluents, excipients and/or stabilizers, are known in the art, and may be selected in accordance with standard practices in the industry (Remington, The Science and Practice of Pharmacy (2000)).

An occludin peptide may be also be conjugated to protein transduction domains (PTDs), small peptides of about 10-16 residues having numerous positively charged lysine (K) and arginine (R) residues, for delivery. Preferably, the PTDs should have between 1-5 such residues. Studies have shown that a functional protein as large as beta-galactosidase (approximately 120 kDa) can be delivered to cells intracellularly via an interperitoneal injection when it is conjugated to Tat-PTD (Tat stands for transactivator protein from lentoviruses) with a sequence of NH2-RKKRRQRRR (Schwarze et al., 1999; Schwarze and Dowdy, 2000). Conjugation with PTDs may enhance efficacy of the contraceptive by improving delivery of the occludin peptide to the Sertoli cells in the testis.

Contraception may also be achieved by administering to the male a nucleic acid encoding the occludin peptide (or the fusion protein) via a suitable delivery system. For example, an adenovirus/retrovirus-mediated gene transfer system may also be used to deliver the peptide. See, Blanchard and Boekelheide, 1997; Nagano et al., 2000. For example, a DNA molecule encoding the peptide corresponding to the occludin peptide linked to a reporter gene such as β-galactosidase is inserted into a replication-defective viral expression vector such as human adenovirus serotype 5 (Stratford-Perricaudet et al., 1990; Lee et al., 1993; Blanchard et al., 1997). In a preferred embodiment, the replication-incompetent human adenovirus serotype 5 containing both E1 and E3 deletions are constructed where the coding sequences of the occludin peptide and reporter gene are inserted and driven by the adenoviral promoter. In vitro viral packaging is performed to produce a high-titer viral stock. The concentration of viral stock is quantified with the 293 human embryonic kidney cells using a plaque forming unit assay (Hitt et al., 1995). The viral stock is then diluted into a proper concentration with PBS containing CaCl₂ and MgCl₂ and delivered into the testis by intratesticular injection.

It is to be understood that while the invention has been described in conjunction with the preferred specific embodiments thereof, that the description above as well as the examples which follow are intended to illustrate and not limit the scope of the invention. Other aspects, advantages and modifications within the scope of the invention will be apparent to those skilled in the art to which the invention pertains.

The following examples describe experimentation performed using a peptide of the present invention which is a 22-amino acid peptide corresponding to the second extracellular domain of rat occludin. The peptide was tested in vitro by measuring transepithelial electrical resistance across Sertoli cell epithelia. The peptide reversibly disrupted Sertoli cell tight junctions. The peptide was also evaluated in vivo by way of testicular injection in male rats, the results of which showed reversible depletion of germ cells from the seminiferous epithelium and disruption of the blood-testis barrier.

Animals. Adult (weighing between 250 and 300 g body weight) and 20-day-old Sprague-Dawley rats were obtained from Charles River Laboratories (Kingston, Mass.). All rats were housed at the Rockefeller University Laboratory Animal Research Center (LARC). Two adult rats were housed per cage. All animals had free access to standard rat chow and water ad libitum under controlled temperature (22° C.) and constant light:dark cycles of 12 hr:12 hr. These animals were maintained in accordance with the applicable portions of the Animal Welfare Act and the guidelines in the Department of Health and Human Services publication: “Guide for the Care and Use of Laboratory Animal”. The use of animals as described in this application was approved by the Rockefeller University Animal Care and Use Committee with Protocol Numbers: 97117, 95129R and 00111.

Preparation of Sertoli cell cultures. Primary Sertoli cells were isolated from 20-day-old Sprague-Dawley rat testes as previously described (Cheng et al., 1986). For low cell density cultures, Sertoli cells were plated at 5×10⁴ cells/cm² in 100-mm Petri dishes [approximately 4.5×10⁶ cells/100-mm dish/9 ml Ham's F12 Nutrient Mixture/Dulbecco's Modified Eagle's Medium (F12/DMEM, 1:1, vol/vol, Life Technologies, Inc.); F12/DMEM were supplemented with 15 mM HEPES, 1.2 g/L sodium bicarbonate, 10 μg/ml bovine insulin, 5 μg/ml human transferrin, 2.5 ng/ml EGF, 20 mg/L gentamicin and 10 μg/ml bacitracin. Sertoli cells cultured under low cell density formed monolayer epithelia without the assembly of inter-Sertoli TJs when monitored by TER measurement (Grima et al., 1998). However, both adherens junctions (AJs) and gap junctions (GJs) were formed (Chung et al., 1999). For high cell density cultures, Sertoli cells were plated on Matrigel™ (Collaborative Biochemical Products, Belford, Mass.)-coated (diluted 1:7 with F12/DMEM, vol/vol) 24-well dishes (effective surface area, approximately 1.88 cm², with 2 ml F12/DMEM per well) at a density of 0.6-3×10⁶ cells/cm² as previously described (Grima et al., 1998) to allow formation of TJs, AJs and GJs, mimicking Sertoli cells found in vivo when assessed by various criteria (Grima et al., 1992). Cell cultures were incubated in a humidified atmosphere of 95% air and 5% CO₂ (vol/vol) at 35° C. and TER readings were recorded 24 hr later and designated as cultures at day 1. These Sertoli cell cultures were shown to have purity greater than 95% when examined microscopically (Wong et al., 2000; Grima et al., 1992; Grima et al., 2000).

Detection of occludin steady-state mRNA level by semi-quantitative RT-PCR. Semi-quantitative RT-PCR was performed essentially as earlier described (Grima et al., 1998; Chung et al., 1998a, b; 1999; Monk and Cheng, 1999). RNA was extracted from cultured cells at specified time points by RNA STAT-60™ (Tel Test “B” Inc., Friendswood, Tex.). Approximately 3 μg of total RNA was reverse-transcribed into cDNAs using 1 μg of oligo-dT₁₅ and a MMLV reverse transcription kit (Promega, Madison, Wis.) in a final reaction volume of 25 μl. In order to quantify and compare the levels of occludin mRNA from various samples, occludin was coamplified with S16 so that the relative expression of occludin could be normalized against S 16. In preliminary experiments, PCR was performed using different concentrations of template and primer pairs, and PCR products were examined over a range of 20-28 amplification cycles to ensure the linearity of the target gene and S16. In most experiments, PCR was performed by combining 3 μl of the RT product with 0.4 μg each of occludin primers (sense primer: 5′-CTGTCTATGCTCGTCATCG-3′, nucleotides 770-788 and antisense primer: 5′-CATTCCCGATCTAATGACGC-3′, nucleotides 1044-1063) (Genebank Acession Number AB016425), 80 ng each of rat ribosomal S16 primers (sense primer: 5′-TCCGCTGCAGTCCGTTCAAGTCTT-3′, nucleotides 15-38 and antisense primer: 5′-GCCAAACTTCTTGGATTCGCAGCG-3′, nucleotides 376-399) (Chan et al., 1990), 5 μl 10X PCR buffer, 3 μl of MgCl₂ (25 mM), 8 μl of dNTPs (200 μM each of dATP, dGTP, dCTP, and dTTP), 2.5 unit Taq DNA polymerase (Promega), and sterile double distilled water to a final volume of 50 μl. The cycling parameters for PCR were as follows: denaturation at 94° C. for 1 min, annealing at 62° C. for 2 min and extension at 72° C. for 3 min for a total of 23 cycles, which was followed by a 15 min extension at 72° C. In order to enhance the detection limit and to yield data for semi-quantitative analysis following densitometric scanning of the resultant autoradiograms, PCR was performed in the presence of [γ-³²P]-labeled primer. Briefly, the sense primer of occludin and S16 were labeled at the 5′-end with γ-³²P]DATP (specific activity, 6000 Ci/mmol, Amersham Pharmacia Biotech) using T₄ polynucleotide kinase (Promega, Madison, Wis.). The relative ratio of the [γ-³²P]-S16(sense primer, ˜10,000 cpm/PCR tube):[γ-³²P]-occludin (sense primer) was the same as the unlabeled corresponding sense primer so that the resultant autoradiograms are the replicate of the ethidium bromide stained gel. About 10 μl aliquots of the PCR product were resolved onto 5% T polyacrylamide gels using 0.5×TBE (45 mM Tris-borate, 1 mM EDTA, pH 8.0) as a running buffer. PCR products were visualized by ethidium bromide staining. Gels were then dried and autoradiography was performed using Kodak X-OMAT AR film (Eastman Kodak, Rochester, N.Y.). The resultant autoradiograms were densitometrically scanned at 600 nm using an UltroScan XL Enhanced Laser Densitometer (Pharmacia Amersham Biotech), and data were normalized against S16 to yield semi-quantitative data.

Synthesis, purification, and characterization of a 22-amino acid occludin synthetic peptide. A 22 amino acid peptide corresponding to the second extracellular domain of rat occludin (NH₂—GSQIYTICSQFYTPGGTGLYVD—COOH, amino acid residues 209-230) (Genebank Acession Number AB016425) and a 22 amino acid myotubularin (NH₂—TKVNERYELCDTYPALLAVPAN—COOH, residues 156-177) (Li et al. 2000a) were obtained from SynPep Corp (Dublin, Calif.). These peptide sequences shared no homology to existing entries at GenBank using BLAST search software. However, the short stretch of sequence for rat occludin shared 90% homology among occludin isolated from different species. To purify the synthetic peptide, 500 μg of the crude peptide was dissolved in solvent A (5% acetonitrile, 95% water, containing 0.1% trifluoroacetic acid, vol/vol) and loaded onto a Vydac™ (Separations Group, Hesperia, Calif.) C18 reverse-phase HPLC column (4.6×250 mm i.d.) at a flow rate of 1 ml/min. The occludin peptide was then separated from other contaminants and eluted using a linear gradient of 15-65% solvent B (95% acetonitrile, 5% water containing 0.1% trifluoroacetic acid, vol/vol) over a period of 30 min as described (Li et al., 2000; Monk & Cheng, 1999). The eluents were monitored by UV absorbance at 220 nm, and fractions of 0.5 ml each were collected. Fractions containing the occludin peptide were pooled, frozen and lyophilized. Thereafter, about 50 pmol of the purified occludin peptide was microsequenced to confirm its identity as previously described (Monk and Cheng, 1999; Cheng et al. 1998; and Cheng et al. 1989). The repetitive yield was about 96%.

Assessing the integrity of the inter-Sertoli TJ-permeability barrier by measuring the transepithelial electrical resistance (TER) across Sertoli cell epithelia To assess the effects of occludin peptide on the assembly of inter-Sertoli TJs, Sertoli cells isolated from 20-day-old rat testes were cultured at 1.2×10⁶ cells/cm² to allow the assembly of inter-Sertoli TJs, and the TER, which is a quantitative measurement of TJ integrity, across the Sertoli cell epithelia was quantified as previously described (Wong et al., 2000; Grima et al. 1998). Cells were plated on Matrigel™ (1:7)-coated HA filters in the apical chamber (Millipore, Bedford, Mass.) (25, 28). Great care was taken so that air bubbles were not trapped between Sertoli cell aggregates, which is the major obstacle to obtain steady TER across the Sertoli cell epithelia since air bubbles create physical pores between adjacent Sertoli cells. TER across the Sertoli cell epithelia at specific time points was determined by a Millicell electrical resistance system as described (Grima et al., 1998; Cheng and Chung, 2001). The resistance in ohm was multiplied by the effective surface area of the bicameral unit (approximately 0.6 cm²) to yield the area resistance (ohm.cm²). The net value of electrical resistance was then computed by substracting the background, which was measured on the Matrigel-coated cell-free chambers, from values of Sertoli cell-plated chambers. To minimize temperature-induced fluctuations during TER measurement, cultures were stabilized at room temperature for 20-30 minutes prior to recording TER across the Sertoli cell epithelia. Synthetic occludin peptide at 0.2-4 μM was included in the basal (0.5 ml F12/DMEM containing 0.03% DMSO, vol/vol, DMSO was used to solubilize the peptide in medium) and apical (0.5 ml F12/DME containing 0.03% DMSO, vol/vol) chambers of the bicameral units 24 hours after freshly isolated Sertoli cells were plated onto Matrigel-coated units (day 1). Peptide was included in F12/DMEM containing 0.03% DMSO (vol/vol) when media were replaced daily. In selected experiments, synthetic occludin peptide was removed from the Sertoli cell epithelia by rinsing cells with two successive washes of F12/DMEM without peptide and subsequent media also contained no peptide. Control experiments included: (i) Sertoli cells cultured alone, (ii) Sertoli cells cultured with vehicle only (media with 0.03% DMSO, vol/vol), and (iii) Sertoli cells cultured in the presence of 4 μM of the 22-amino acid synthetic myotubularin peptide as described above. Each time point contained triplicate cultures, and each experiment was repeated 2-3 times using different batches of Sertoli cells. We have selected TER measurement to quantify and assembly and maintenance of inter-Sertoli TJs over other methodologies, which include: (i) restriction of diffusion of [³H]-inulin, [¹²⁵I]-BSA or fluorescein isothiocyanate-labeled dextran across the Sertoli cell epithelia, (ii) maintenance of nonequilibrium of the media in the apical and basal chamber of the bicameral units, and (iii) polarized secretion of Sertoli cell products such as transferrin, rABP, testin, clusterin, and α2-macroglobulin, as described (Grima et al., 1992; Grima et al., 2000), for the following reasons. First, this is a technique widely adopted by cell biologists in the field (Wong et al. 1997; Balda et al. 1996). Second, it yields quantitative measurement on the assembly and maintenance of inter-Sertoli TJs. Third, and most important, results obtained by TER measurement are consistent with other tedious approaches as described above, such as restriction diffusion of [³H]-inulin and fluorescein isothiocyanate-labeled dextran (monitored by a Tecan GENios cytofluorometer) was used in parallel with TER to assess the tight junction permeability barrier.

Intratesticular injection of occludin peptide and histological analysis of the testis. To assess the in vivo effects of the occludin peptide on spermatogenesis, peptide was administered to testes of adult rats by direct intratesticular injection as follows. Peptide was suspended in 0.9% sterile saline, sterilized by exposure to UV for 5 minutes. It was noted that this brief UV treatment to sterilize the peptide suspension prior to its use did not alter its structure and was verified by two approaches. First, UV treated peptide retained the same retention time on reverse-phase HPLC using a Vydac C18 column (4.6×250 mm, i.d.) (Cheng et al., 1998; Cheng et al., 1989) when compared to peptide prior to the UV treatment. Second, the primary sequence of the UV-trated peptide remain unaltered when direct protein microsequencing was performed as described (Mruk and Cheng, 1990; Cheng et al., 1998; Cheng et al., 1989). Adult rats weighing between 250 and 300 g body weight were anesthetized with Metofane® before treatment. Rats received either 300 μl of 0.9% sterile saline (vehicle control), no treatment (control), or 1.5-10 mg of occludin peptide suspended in 300 μl of 0.9% sterile saline intratesticularly. The right testis of an animal received the peptide or vehicle and the left testis of the same animal was not treated and was used as a control. Peptide or vehicle was administered at 3 sites/testis with about 100 μl sample per site essentially as previously described (Grima et al., 1998; Eng et al., 1994). In another control group, rats were injected with a synthetic 22-amino acid peptide of NH₂—TKVNERYELCDTYPALLAVPAN—COOH based on a known Sertoli cell protein, rat myotubularin (rMTM) (Li et al., 2000; Li et al., 2001), which had no sequence homology with occludin. Three rats were used in each treatment group and rats were sacrificed by CO₂ asphyxiation at specific time points. Testes were removed immediately and fixed in 10% neutral buffered formalin. Testes were embedded in paraffin, and dehydrated in graded ethanol. For morphological analysis, 5 μm sections were cut and stained with hematoxylin and eosin. About 50 sections were examined at different sites for each testis using an Olympus BX40 microscope (Tokyo, Japan), interfaced to an Olympus PM-30 Exposure Control Unit.

Assessing the occludin-peptide induced disruption of the blood-testis barrier (BTB) by micropuncture techniques.

Radioiodination of bovine serum albumin (BSA).

Briefly, 5 μg of BSA (Sigma, RIA grade, Mr 68 kDa) was radioiodinated by Iodogen (Fraker et al., 1978) using 1 mCi of [¹²⁵I]-sodium iodide (Amersham Pharmacia Biotech) as described (Cheng et al., 1983).

Detection of [¹²⁵]-BSA in seminiferous tubular fluid (STF) and rete testis fluid (RTF). 2, 4, 6, and 12-week after an intratesticular administration of 1.5 mg of either occludin or myotubularin peptide/testis as described above (administered at 3 sites to the right testis) where the left testis of the same animal was used as a control. Rats (n=4-6 rats per time point, approximately 250 gm b.w. at the time of peptide treatment) were anesthetized with ketamine HCl (Fort Dodge Laboratories, Inc., Fort Dodge Iowa) at 60 mg/kg b.w. Micropuncture was performed essentially as described (Turner et al., 1984; Stahler et al., 1991). Briefly, testes were exposed through an abdominal incision, and the efferent ducts were ligated with surgical silk thread. Testes were then returned to the scrotum, wound cleansed with 70% ethanol, surgically closed and animals were allowed to recover. Twenty-four hours after efferent duct ligation, rats were anesthetized by ketamine HCl, bilateral nephrectomy was performed to prevent renal excretion of [¹²⁵I]-BSA, and approximately 6×10⁶ cpm of [¹²⁵I]-BSA was infused into the rat via jugular vein. Two hours after infusion, testes were removed, RTF and STF were collected as described (Turner et al, 1984; Stahler et al., 1991) for radioactivity determination in a γ-counter. The left testis from the same animal without receiving either the occludin or myotubularin peptide served as a control, and both STF and RTF were also collected for radioactivity determination to assess the integrity of the BTB.

Statistical analysis. Results were analyzed for statistical significance either by Student's t-test to compare treated samples with their corresponding controls or by ANOVA using the GB-STAT Statistical Analysis Package (Version 7.0, Dynamic Microsystems Inc., Silver Spring, Md.). Using Tukey's honestly significant difference (HSD) test for ANOVA, results of individual samples were compared to controls and to samples subjected to the same treatment within the same group. In all culture experiments for studying cellular gene expression or for TER measurement to assess inter-Sertoli TJ permeability barrier, each time point had replicate cultures and each experiment was repeated 2-3 times using different batches of Sertoli cells.

Expression of occludin by Sertoli cells correlates with the assembly of inter-Sertoli TJ-permeability barrier in vitro

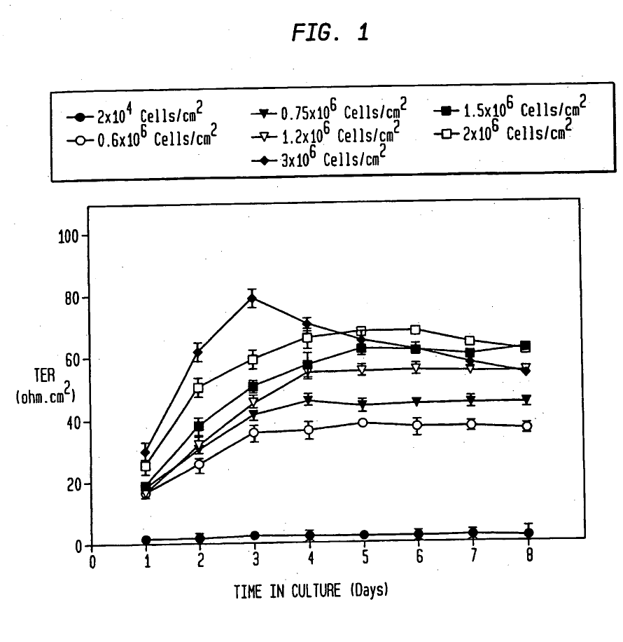

When Sertoli cells were cultured at different cell densities ranging between 2×10⁴ and 3×10⁶ cells/cm² on Matrigel-coated bicameral units, a steady increase in TER across the Sertoli cell epithelia was noted (FIG. 1). The assembly of inter-Sertoli TJs were completed by day 4 as manifested by a stable TER across the Sertoli cell epithelia (FIG. 1). These results were consistent with other techniques used to assess the inter-Sertoli TJ permeability barrier, such as restricted diffusion of [³H]-inulin across the Sertoli cell epithelia, polarized secretion of Sertoli cell secretory products, such as rABP, transferrin, and testing (Grima et al., 1992; Byers et al., 1986; and Janecki et al., 1986). Since TER measurement yields quantitative assessment on the inter-Sertoli TJ assembly, it does not require the use of radioactive isotopes; the method is highly reproducible and is relatively easy to set up and maintain. In addition, it is widely used by cell biologists in the field (Wong et al., 1997; Balda et al., 1996). Accordingly, this method was thus selected over other approaches.

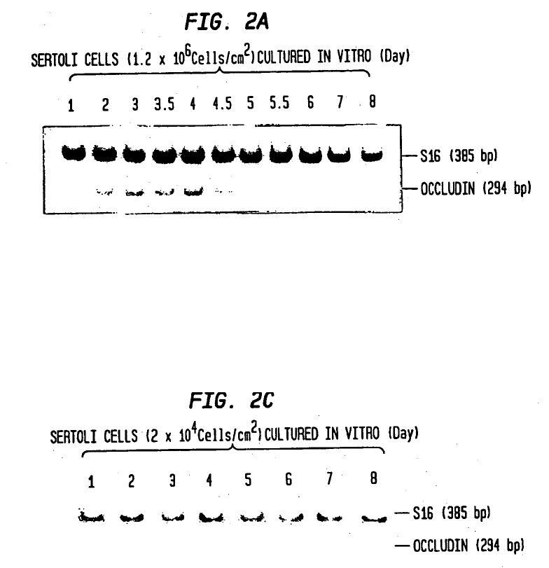

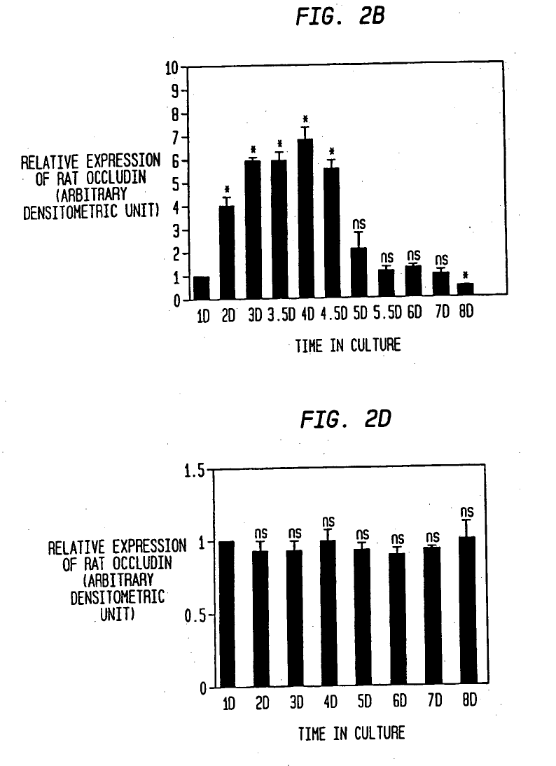

Sertoli cells cultured on Matrigel-coated bicameral units at different cell densities displayed a similar pattern of TER profile over time in culture during TJ assembly but the “tightness” of the inter-Sertoli TJ positively correlated with the cell density. When Sertoli cells cultured at 3×10⁶ cells/cm², there was a mild decline in TER after day 4 in three different experiments. This may be a result of cell overcrowding and death, accumulation of metabolic wastes, and insufficient nutrient flow. Morphological studies have shown that cells cultured at such high density are accompanied by an increase in DNA fragmentation derived from degenerating Sertoli cells (Wong et al., 2000). At low cell density (2×10⁴ cells/cm²), no measurable TJ permeability was detected, possibly due to the lack of close cell proximity to allow TJ assembly because of insufficient cell number (FIG. 1). At high cell density (1.2×10⁶ cells/cm²), there was a significant but transient increase in occludin steady-state mRNA level between days 2 and 4.5 (FIGS. 2A,B) at the time when inter-Sertoli TJs were assembled (FIG. 1 versus FIG. 2), illustrating that TJ assembly required de novo synthesis of occludin, which was one of the building blocks of the TJs. After day 5, the steady-state occludin mRNA returned to the basal level similar to day 1 (FIG. 2A, B). Such a transient induction in occludin expression was not detected in low cell density cultures at 2×10⁴ cells/cm² (FIG. 2C versus FIGS. 2A,B). These results were consistent with previous observations showing a correlation between ZO-1 induction, also a TJ-associated protein, and inter-Sertoli TJ assembly (Chung et al., 1999; Wong et al., 2000).

Reversible perturbation of inter-Sertoli TJ-permeability barrier in vitro by the use of a 22-amino acid synthetic peptide corresponding to a segment of the second external loop of occludin

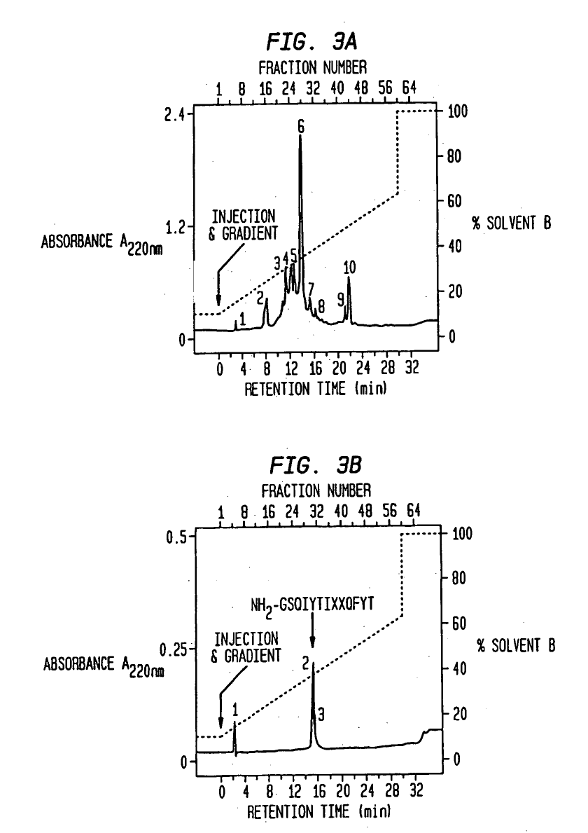

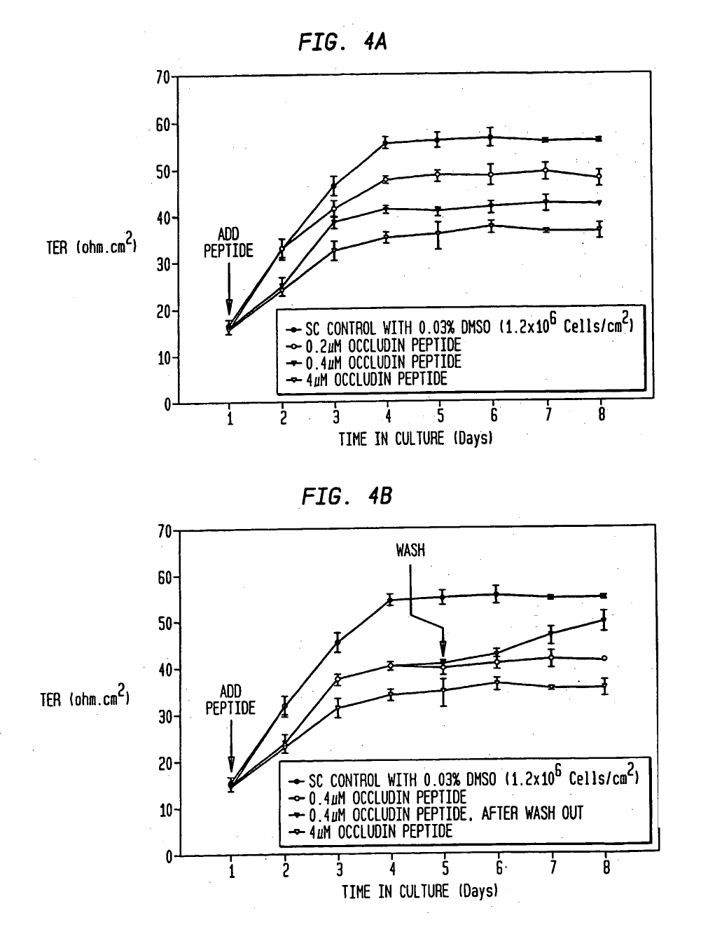

A 22-amino acid synthetic peptide corresponding to the outermost region of the second external loop of rat occludin (residues 209-230 on Table 1) was assessed for its ability to affect the assembly of inter-Sertoli TJs. Following its synthesis, the synthetic occludin peptide was purified by reverse-phase HPLC (FIGS. 3A,B) and its identity was confirmed by direct protein microsequencing. Addition of occludin peptide to the Sertoli cell epithelia 24 hours after isolation at a density of 1.2×10⁶ cells/cm² induced a dose-dependent decline in TER (FIG. 4A). This peptide-induced disruption of paracellular permeability barrier could be reversed after its removal from the culture (FIG. 4B). Sertoli cells incubated with occludin peptide at 4 μM caused a significant decline in TER, approximately 50% when compared to untreated controls on days 4-5. In selected experiments, when the occludin peptide was removed on day 5 from the bicameral units by two successive washes using F12/DMEM, the inter-Sertoli TJ permeability barrier could be reassembled making the TER reading indistinguishable from controls within 3-4 days (FIG. 4B). Interestingly, the time it took to reassemble the disrupted inter-Sertoli TJ induced by the occludin peptide was roughly equivalent to that of the inter-Sertoli TJ assembly using freshly isolated Sertoli cells in vitro, which was different from the [Ca²⁺] depletion-induced TJ leakiness as it took the Sertoli cell only 90 minutes to reseal the disrupted TJ (Grima et al., 1998). Since the effects of occludin peptide in perturbing the inter-Sertoli TJ permeability barrier were reversible after the peptide removal, it was determined that the occludin peptide was non-toxic to the Sertoli cells. When a 22-amino acid rMTM peptide at 4 μM was used instead of the occludin peptide, it had no apparent effects to perturb the inter-Sertoli TJ barrier (data not shown). Also, when the cell viability in the occludin peptide-treated cultures was assessed by trypan blue dye-exclusion test versus control cultures in selected experiments, no apparent differences were detected (data not shown). Other polypeptides obtained from the second extracellular loop of occludin (or analogs thereof) may be readily evaluated for effectiveness by carrying out the foregoing test.

Reversible effects of occludin peptide on spermatogenesis in vivo

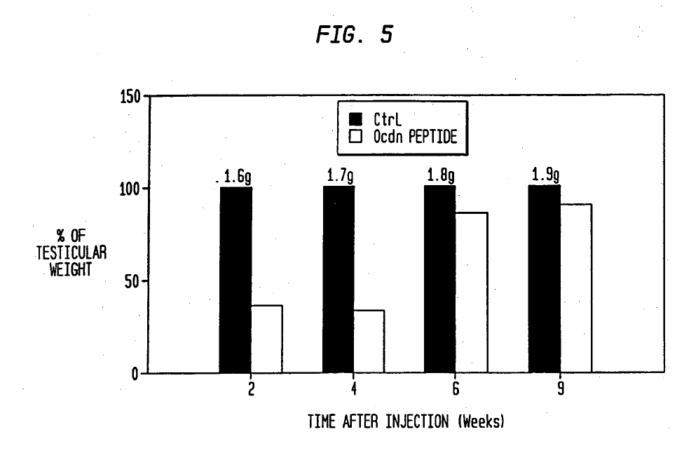

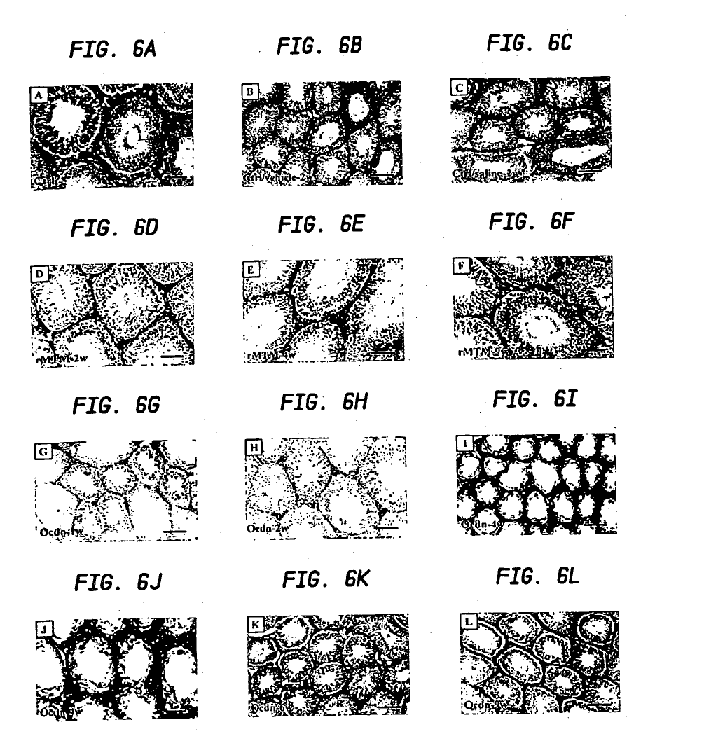

Following HPLC purification, occludin or myotubularin peptide was suspended in 0.9% sterile saline and sterilized under UV for 5 minutes. Each rat in the experimental group received an intratesticular injection of 300 μl of saline containing either 0.15 mg or 1.5 mg of the corresponding purified peptide. The peptide suspension was distributed in each testis at 3 sites (approximately 100 ml per site) as described in Materials and Methods and detailed elsewhere (Grima et al., 1992; Eng et al., 1994). Intratesticular injection of this purified occludin peptide caused a reduction in testicular weight within 2 weeks when compared to control rats that received no treatment, vehicle (0.9% sterile saline) alone, or myotubularin peptide (FIG. 5). While there was an occludin peptide induced decline in testicular weight and testicular size (FIG. 5), the appearance of the testis and the epididymis appeared normal. FIG. 6A-C showed the control rat testes received either no treatment (FIG. 6A), 14 days later after an intratesticular injection of vehicle alone (FIG. 6B) or 14 days later after an intratesticular injection of saline (FIG. 6C). FIGS. 6D-F are another control set of testes where rats received an intratesticular injection of the myotubularin peptide at 10 (FIG. 6D), 23 (FIG. 6E), and 40 days (FIG. 6F) post-treatment. Morphological analysis of the treated testis revealed that more advanced germ cells, such as elongated spermatids, began to deplete from the epithelium between 8 (FIG. 6G) and 16 days after the occludin peptide treatment. Massive depletion of germ cells from the epithelium in virtually all the tubules examined was noted by 27 days after intratesticular occludin peptide injection (FIGS. 6I and 6J). In addition, the seminiferous tubules of the occludin-treated testes shrunk significantly with the tubular diameter reduced by as much as 20-30% when compared to control rats or testes which received only vehicle or myotubularin peptide (FIGS. 6I and 6J versus 6A-C). Germ cells began to repopulate in the epithelium after 27 days post occludin peptide treatment. By 47 days, spermatocytes were clearly visible in all the tubules examined (FIG. 6K), and the morphology of the seminiferous epithelium appeared indistinguishable from control rats by 68 days post occludin peptide treatment (FIG. 6L), showing full recovery from the occludin peptide-induced damage in the testes (FIG. 6L). The fact that the testes recovered almost fully within 40 days (FIG. 6L at 68 days versus FIGS. 6I, J at 27 days post-treatment) suggests that most of the spermatocytes were not destroyed by the occludin peptide treatment.

Effects of occludin peptide on the blood-testis barrier (BTB)

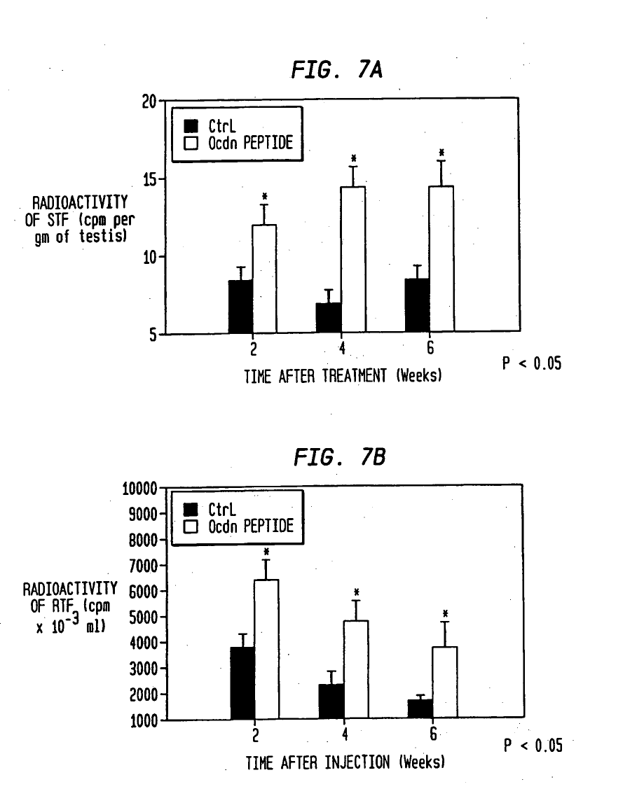

To determine whether the peptide-induced germ cell loss was due to a disruption of the BTB (which in turn induces the host immune system to mount an attack causing germ cell resorption, such as those shown in FIG. 6) the integrity of the BTB was assessed following an intratesticular injection of synthetic peptide (either occludin or myotubularin peptide) at three sites per testis, while the other testis of the same animal (adult Sprague-Dawley rats, weighing between 270-300 gm) was used as a control (n=4-6 rats per time point). Results shown in FIG. 7 clearly illustrated a disruption of the BTB following an intratesticular injection of occludin peptide at 1.5 mg/testis. There was an accumulation of [¹²⁵I]-BSA in the STF (FIG. 7A) and RTF (FIG. 7B) in the occludin peptide-injected testes between 2-6 weeks post-treatment compared to the untreated testes in the same rats after infusion of [¹²⁵I]-BSA through the jugular vein. The peptide-induced damage to BTB appeared to be reversible since there was a drastic decline in [¹²⁵I]-BSA accumulation in both STF and RTF (FIG. 7A,B) by 12 weeks post-treatment coinciding with the recovery of the seminiferous epithelium when examined by histological analysis (data not shown) similar to those shown in FIG. 6. Moreover, the level of radioactivity in the STF and RTF collected from peptide-treated rats became indistinguishable from control testes, which had not exposed to the occludin peptide (FIG. 7). This occludin peptide-induced damage to the BTB appears to be specific since the 22 amino acid rat myotubularin peptide failed to induce disruption of the BTB because the radioactivity detected in either the STF or RTF in myotubularin peptide-treated rats was indistinguishable from control rats which received no peptide treatment (FIGS. 7A, B).

Preparation of cDNAs encoding for wild type FSH subunits and corresponding mutants by PCR

Table 3 shows full-length cDNAs encoding for the α and β subunits of follicle stimulating hormone (“FSH”) for the wild type, two mutants for FSH-α, and one mutant for FSH-β. Table 4 shows three recombinant FSH mutants which can be prepared using the baculovirus system as described in Sorrentino et al., 1998. To obtain these cDNAs, site-directed mutagenesis using PCR can be performed as described in McPherson, 1991. Briefly, total RNAs are isolated from rat pituitary glands for the RT reaction using oligo(dT)15. These cDNAs serve as templates for subsequent PCR. Appropriate primer pairs containing the mutated sites are synthesized, which are used for PCR using the above RT product as a template as described (McPherson, 1991; Sorrentino et al., 1998). Recombinant FSH proteins lacking the putative glycosylation sites (see. Tables 3 and 4), which are known to contribute to the overall hormonal activity of FSH (Rose et al., 2001), are prepared by converting Asn (N) at 56 and 82 from the N-terminus to Asp (D) (see Table 3 and Mutant 1 in Table 4). Further, mutants are prepared by replacing Thr-Met-Leu (residues 50-52) and Met-Gly-Asn (residues 75-77) with Lys-Lys-Lys and Ala-Ala-Ala, respectively (see Table 3 and Mutant 4 in Table 2). The objective is to alter the hydrophobicity and conformation on sites near the putative glycosylation sites. A third mutant consists of only the FSH-β with a deletion at the putative glycosylation site (see Table 3 and Mutant 4 in Table 2). PCR products following site-directed mutagenesis experiments encoding for the three different FSH-α and two FSH-β subunits shown in Tables 3 and 4, are then subcloned in pGEM-T vector (Promega) for direct nucleotide sequence analysis to confirm the sequence at the desired mutated sites. cDNA containing the desired mutated sites can then be ligated to a plasmid, for example, pAcGzt, which may be used for transfection into baculovirus for recombinant protein production.

TABLE 3

Amino acid sequences of the α and β subunits of wild
type FSH and FSH mutants.

| | |
|---|---|
| α-Subunit | |
| rFSH α^{WT} | NH₂-MDCYRRYAAVILVMLSMVLHILHSLPDGDLIIQ |
| | GCPECKLKENKYFSKLGAPIYQCMGCCFSRAYPTPARS |
| | KKTMLVPKNITSQATCCVAKSFTKATVMGNARVENHT |
| | DCHCSTCYYHKS-COOH |
| ΔrFSH α-(56/82) | NH₂-MDCYRRYAAVILVMLSMVLHILHSLPDGDLIIQ |
| | GCPECKLKENKYFSKLGAPIYQCMGCCFSRAYPTPARS |
| | KKTMLVPK [symbol] DITSQATCCVAKSFTKATVMGNARVE [symbol] DHT |
| | DCHCSTCYYHKS-COOH |
| ΔrFSH α-(50-52/75-77) | NH₂-MDCYRRYAAVILVMLSMVLHILHSLPDGDLIIQ |
| | GCPECKLKENKYFSKLGAPIYQCMGCCFSRAYPTPARS |
| | KK [symbol] KKKVPKNITSQATCCVAKSFTKATV [symbol] AAAARVENHT |
| | DCHCSTCYYHKS-COOH |
| B-Subunit | |
| rFSH β^{WT} | NH₂-MMKSIQLCILLWCLRAVCCHSCELTNITISVEKEE |
| | CRFCISINTTWCEGYCYTRDLVYKDPARPNTQKVCTFK |
| | ELVYETIRLPGCARHSDSLYTYPVATECHCGKCDSDSTD |
| | CTVRGLGPSYCSFGEMKE-COOH |
| ΔrFSH β-(22-24) | NH₂-MMKSIQLCILLWCLRAVCCHSCELTNITISVEKEE |
| | CRFCIS- - -TWCEGYCYTRDLVYKDPARPNTQKVCTFKE |
| | LVYETIRLPGCARHSDSLYTYPVATECHCGKCDSDSTD |
| | CTVRGLGPSYCSFGEMKE-COOH |

*Full-length nucleotide sequences encoding the corresponding α and β subunits of rat FSH, and mutants thereof, are shown. Amino acid residues in bold represent the signal peptide. Amino acid residues in underline format represent the putative glycosylation sites known to confer hormonal activity of FSH. Residues in bold italics represent the proposed mutation. A broken line “- - -” represents a deletion.

TABLE 4
Proposed recombinant FSH mutants and the wild type (control).

| | FSH α-subunit | FSH β-subunit |
|---|---|---|
| Control | rFSH α^{WT} | rFSH β^{WT} |
| Mutant 1 | ΔrFSH α-(56/82) | ΔrFSH β-(22-24) |
| Mutant 2 | ΔrFSH α-(50-52/75-77) | ΔrFSH β-(22-24) |
| Mutant 3 | No α subunit | ΔrFSH β-(22-24) |

### INDUSTRIAL APPLICABILITY

The invention relates to the medical, veterinary and pharmaceutical industries, particularly to the field of contraception.

All publications mentioned in the specification are indicative of the level of those skilled in the art to which this invention pertains. All of these publications are herein incorporated by reference to the same extent as if each individual publication was specifically and individually indicated to be incorporated by reference.

The foregoing description of the specific embodiments fully reveals the general nature of the invention such that others can, by applying current knowledge, readily modify and/or adapt for various application such specific embodiments without departing from the general nature of the invention. Therefore, such adaptations and modifications are intended to be comprehended within the meaning and range of equivalents of the disclosed embodiments. Additionally, the terminology herein is for the purpose of description and not of limitation.

### CITATION OF PUBLICATIONS CITED HEREIN

1. de Kretser DM, Kerr JB. The cytology of the testis. In The Physiology of Reproduction, Vol. 1. Eds. Knobil E, Neil JD, Ewing LL, Greenwald GS, Markert CL, Pfaff DW. Raven Press, New York. 1988; pp. 837-932.

2. Russell LD, Ettlin RA, Sinha Hikim AP, CLEGG EJ (Eds.) Histological and Histopathological Evaluation of the Testis. Clearwater, Cache River Press, 1990; p. 52.

3. Mruk D, Cheng CY. Sertoli cell proteins in testicular paracriny. In Testis, Epididymis and Technologies in the year 2000. Eds. Jegou B, Pineau C, Saez J. Springer-Verlag, Berlin. 2000; pp. 197-228.

4. Chung SSW, Lee WM, Cheng CY. Study on the formation of specialized inter-Sertoli cell junction in vitro. J Cell Physiol 1999; 181:258-272.

5. Wong CCS, Chung SSW, Grima J, Zhu LJ, Mruk D, Lee WM, Cheng CY. Changes in the expression of junctional and nonjunctional complex component genes when inter-Sertoli tight junctions are formed in vitro. J Androl 2000; 21:227-237.

6. Lui WY, Lee WM, Cheng CY. Transforming growth factor-13 perturbs the inter-Sertoli tight junction permeability barrier in vitro possibly mediated via its effects on occludin, zonula occludens-1, and claudin-11. Endocrinology 2001; 142:1865-1877.

7. Samy ET, Li JCH, Grima J, Lee WM, Silvestrini B, Cheng CY. Sertoli cell prostaglandin D₂ synthetase is a multifunctional molecule: its expression and regulation. Endocrinology 2000; 141:710-721.

8. Li JCH, Samy ET, Grima J, Chung SSW, Mruk D, Lee WM, Silvestrini B, Cheng CY. Rat testicular myotubularin (rMTM), a protein tyrosine phosphatase expressed by Sertoli and germ cells, is a marker to study cell-cell interactions in the rat testis. J Cell Physiol 2000; 185:366-385.

9. Li JCH, Lee WM, Mruk D, Cheng CY. Regulation of Sertoli cell myotubularin (rMTM) expression by germ cells in vitro. J Androl 2001; 22:266-277.

10. Furuse M, Fujita K, Hiiragi T, Fujomoto K, Tsukita S. Claudin-1 and -2: novel integral membrane proteins localizing at tight junctions with no sequence homology to occludin. J Cell Biol 1998; 141:1539-1550.

11. Morita K, Furuse M, Fujimoto K, Tsukita S. Claudin multigene family encoding four-transmembrane domain protein components of tight junction strands. Proc Natl Acad Sci USA 1999; 96:511-516.

12. Morita K, Sasaki H, Fujimoto K, Furuse M, Tsukita S. Claudin-11/OSP-based tight junctions of myelin sheaths in brain and Sertoli cells in testis. J Cell Biol 1999; 145:579-588.

13. Martin-Padura L Lostaglio S, Schneemann M, Williams L, Romano M, Fruscella P, Panzeri C, Stoppacciaro A, Ruco L, Villa A, Simmona D, Dejana E. Junctional adhesion molecule, a novel member of the immunoglobulin superfamily that distributes at intercellular junctions and modulates monocytes transmigration. J Cell Biol 1998; 142:117-127.

14. Furuse M, Hirase T, Itoh M, Nagafuchi A, Yonemura S, Tsukita S. Occludin: a novel integral membrane protein localizing at tight junctions. J Cell Biol 1993; 123:1777-1788.

15. Fujimoto K Freeze-fracture replica electronic microscopy combined with SDS digestion for cytochemical labeling of integral membrane proteins. Application to the immunogold labeling of intercellular junctional complexes. J Cell Sci 1995; 108:3443-3449.

16. Saitou M, Ando-Akatsuka Y, Itoh M, Furuse M, Inazawa J, Fujimoto K, Tsukita S. Mammalian occludin in epithelial cells: its expression and subcellular distribution. Eur J Cell Biol 1997; 73: 222-231.

17. Tsukita S, Furuse M. Overcoming barriers in the study of tight junction functions: from occludin and claudin. Genes Cells 1998; 3: 569-573.

18. Fanning AS, Mitic LL, Anderson JM. Transmembrane proteins in the tight junction barrier. J Am Soc Nephrol 1999; 10:1337-1345.

19. Mitic LL, Van Itallie CM, Anderson JM. Molecular physiology and pathophysiology of tight junctions I. Tight junction structure and function: lessons from mutant animals and proteins. Am J Physiol 2000; 279:G250-G254.

20. Ando-Akatsuka Y, Saitou M, Hirase T, Kishi M, Sakakibara A, Itoh M, Yonemura S, Furuse M, Tsukita S. Interspecies diversity of the occludin sequence: cDNA cloning of human, mouse, dog, and rat-kangaroo homologues. J Cell Biol 1996; 133: 43-47.

21. Van Itallie CM, Anderson JM. Occludin confers adhesiveness when expressed in fibroblasts. J Cell Sci 1997; 110:1113-1121.

22. Wong V, Gumbiner BM. A synthetic peptide corresponding to the extracellular domain of occludin perturbs the tight junction permeability barrier. J Cell Biol 1997; 136:399409.

23. Cheng CY, Mather JP, Byer AL, Bardin CW. Identification of hormonally responsive proteins in primary Sertoli cell culture medium by anion-exchange high performance liquid chromatography. Endocrinology 1986; 118:480488.

24. Grima J, Wong CS, Zhu LJ, Zong SD, Cheng CY. Testin secreted by Sertoli cells is associated with the cell surface, and its expression correlates with the disruption of Sertoli-germ cell junctions but not the inter-Sertoli tight junction. J. Biol. Chem. 1998; 273: 21040-21053.

25. Grima J, Pineau C, Bardin CW, Cheng CY. Rat Sertoli cell clusterin, α₂-macroglobulin, and testin: biosynthesis and differential regulation by germ cells. Mol Cell Endocrinol 1992; 89:127-140.

26. Grima J, Cheng CY. Testin induction: The role of cyclic 3′,5′-adenosine monophosphate/protein kinase A signaling in the regulation of basal and lonidamine-induced testin expression by rat Sertoli cells. Biol Reprod 2000; 63:1648-1660.

27. Chung SSW, Mo MY, Lee WM, Cheng CY. Rat testicular N-cadherin: its cDNA cloning and regulation. Endocrinology 1998; 139:1853-1862.

28. Mruk D, Cheng CY. Sertolin is a novel gene marker of cell-cell interactions in the rat testis. J Biol Chem 1999; 274:27056-27068.

29. Chan YL, Paz V, Olvers J, Wool IG. The primary structure of rat ribosomal protein S16. FEBS Letts 1990; 263:85-88.

30. Cheng CY, Mathur PP, Grima J. Structural analysis of clusterin and its subunits in ram rete testis fluid. Biochemistry 1998; 27:4079-4088.

31. Cheng CY, Grima J, Stahler MS, Lockshin RA, Bardin CW. Testin is structurally related Sertoli cell proteins whose secretion is tightly coupled to the presence of germ cells. J Biol Chem 1989; 264:21386-21393.

32. Eng F. Wiebe JP, Alima LH. Long-term alternations in the permeability of the blood-testis barrier following a single intratesticualr injection of dilute aqueous glycerol. J Androl 1994; 15:311-317.

33. Fraker PJ, Speck JC Jr. Protein and cell membrane iodinations with a sparingly soluble chloramide 1,3,4,6-tetrachloro-3a,6a-diphenylglycoluril. Biochem Biophys Res Commun 1978; 80:849-857.

34. Cheng CY, Bardin CW, Musto NA, Gunsalus GL, Cheng SL, Ganguly M. Radioimmunoassay of testosterone-estradiol-binding globulin in humans: A reassessment of normal values. J Clin Endocrinol Metab 1983; 56:68-75.

35. Turner TT, Jones CE, Howards SS, Ewing LL, Zegeye B, Gunsalus GL. On the androgen microenvironment of maturing spermatozoa. Endocrinology 1984; 115:1925-1932.

36. Stahler MS, Schlegel P, Bardin CW, Silvestrini B, Cheng CY. α2-Macroglobulin is not an acute-phase protein in the rat testis. Endocrinology 1991; 128:2805-2814.

37. Byers SW, Hadley MA, Djakiew D, Dym M. Growth and characterization of polarized monolayrs of epithelial cells and Sertoli cells in dual environment culture chambers. J Androl 1986; 7:59-68.

38. Janecki A, Steinberger A. Polarized Sertoli cell functions in a new two-compartment culture system. J. Androl. 1986; 7:69-71.

39. Balda MS, Whitney JA, Flores C, Gonzalez M, Cereijido M, Balda MS, Gonzalez-Mariscal L, Macias-Silva M, Torres-Marquez ME, Garcia Saniz JA, Cereijido M, Matter K Functional dissociation of paracellular permeability and transepithelial electrical resistance and disruption of the apical-basolateral intramembrane diffusion barrier by expression of a mutant tight junction protein. J Cell Biol 1996; 134: 1031-1049.

40. Dym M, Fawcett DW. The blood-testis barrier in the rat and the physiological compartmentation of the seminiferous epithelium. Biol Reprod 1970; 3:308-326.

41. Pelletier RM, Okawara Y, Vitale ML, Anderson JM. Differential distribution of the tight-junction-associated protein ZO-1 isoforms α(+) and α(−) in guinea pig Sertoli cells: a possible association with F-actin and γ-actin. Biol. Reprod. 1997; 57:367-376.

42. Byers S, Graham R, Dai HN, Hoxter B. Development of Sertoli cell junctional specializations and the distribution of the tight-junction-associated protein ZO-1 in the mouse testis. Am J Anat 1991; 191:3547.

43. Moroi S, Saitou M, Fujimoto K, Sakakibara A, Furuse M, Yoshida 0, Tsukita S. Occludin is concentrated at tight junctions of mouse/rat but not human/guinea pig Sertoli cells in testes. Am J Physiol 1998; 274:C1708-C1717.

44. Tsukita S, Furuse M. Pores in the wall: Claudins constitute tight junction strands containing aqueous pores. J Cell Biol 2000; 149:13-16.

45. Muresan Z, Paul DL, Goodenough DA. Occludin 1B, a variant of the tight junction protein occludin. Mol Biol Cell 2000; 11: 627-634.

46. Cyr DG, Hermo L, Egenberger N, Mertineit C, Transler JM, Laird DW. Cellular immunolocalization of occludin during embryonic and postnatal development of the mouse testis and epididymis. Endocrinology 1999; 140: 3815-3825.

47. Furuse M, Itoh M, Hirase T, Nagafuchi A, Yonemura S, Tsukita S. Direct association of occludin with ZO-1 and its possible involvement in the localization of occludin at tight junctions. J Cell Biol 1994; 127:1617-1626.

48. Fanning AS, Jameson BJ, Jesaitis LA, Anderson JM. The tight junction protein ZO-1 establishes a link between the transmembrane protein occludin and the actin cytoskeleton. J Biol Chem 1998; 273: 29745-29753.

49. Sakakibara S, Furuse M, Saitou M, Ando-Akatsuka Y, Tsukita S. Possible involvement of phosphorylation of occludin in tight junction formation. J Cell Biol 1997; 137:1393-1401.

50. Wong V. Phosphorylation of occludin correlates with occludin localization and formation at the tight junction. Am J Physiol 1997; 273:C1859-C1867.

51. Matter K, Balda MS. Occludin and the functions of tight junctions. Int Rev Cytol 1999; 186:117-146.

52. Mitic LL, Anderson JM. Molecular structure of tight junctions. Annu Rev Physiol 1998; 60: 121-142.

53. McCarthy KM, Skare IB, Stankewich MC, Furuse M, Tsukita S, Rogers RA, Lynch RD, Schneeberger EE. Occludin is a functional component of the tight junction. J Cell Sci 1996; 109: 2287-2298.

54. Saitou M, Fujimoto K, Doi Y, Itoh M, Fujimoto T, Furuse M, Takano H, Noda T, Tsukita S. Occludin-deficient embryonic stem cells can differentiate into polar epithelial cells bearing tight junctions. J Cell Biol 1998; 141:397-408.

55. Chen YH, Merzdorf C, Paul DL, Goodenough DA. COOH terminus of occludin is required for tight junction barrier function in early Xenopus embryos. J Cell Biol 1997; 138: 891-899.

56. Furuse M, Fujimoto K, Sato N, Hirase T, Tsukita S. Overexpression of occludin, a tight junction-associated integral membrane protein, induces the formation of intracellular multicellular bodies bearing tight junction-like structures. J Cell Biol 1996; 109:429-435.

57. Lacaz-Vieira F, Jaeger MMM, Farshori P, Karcha B. Small synthetic prptides homologus to segements of the first external loop of occludin impair tight junction resealing. J Membr Biol 1999; 168: 289-297.

58. Paulsen CA, Christensen RB, Bagatell, CJ. Status of male contraception: hormonal approach. In Fertility Control 2^{nd} Edition. Eds. Corson SL, Derman RJ, Tyrer LB. Goldin Publishers, London. 1994; pp. 281-292.

59. Frick J, Bartsch F, Weiske WH. The effect of monthly depot medroxyprogesterone acetate and testosterone on human spermatogenesis. I. Uniform dosage level. Contraception 1977; 15:649-668.

60. Chung NPY, Cheng CY. Is cadmium chloride-induced inter-Sertoli tight junction permeability barrier disruption a suitable in vitro model to study the events of junction disassembly during spermatogenesis in the rat testis? Endocrinology 2001; 142:1878-1888.

61. Wang W, Uzzau S, Goldblum SE, Fasano A. Human zonulin, a potential modulator of intestinal tight junctions. J Cell Sci 2000; 113:4435-4440.

62. Fasano A, Not T, Wang W, Uzzau S, Berti I, Tommasini A, Goldblum SE. Zonulin, a newly discovered modulator of intestinal tight junctions, and its expression in coeliac disease. Lancet 2000; 355:1518-1519.

63. McPherson MJ (Ed.) Directed mutagenesis. New York, IRL Press, 1991.

64. Rose MP, Das REG and Balen All (2001) Definition and measurement of follicle stimulating hormone Endocrine Rev. 21:5-22.

65. Sorrentino C, Silvestrini B, Braghiroli L, Chung SSW, Giacomelli 5, Leone MG, Xie YB, Sui YP, Mo MY and Cheng CY (1998) Rat prostaglandin D2 synthetase: Its tissue distribution, changes during maturation, and regulation in the testis and epididymis. Biol. Reprod 59:843-853.

66. Schwarze SR, Ho A, Vocero-Akbani A, and Dowdy SF (1999) In vivo protein transduction: delivery of biologically active protein into the mouse. Science 285:1569-1572.

67. Schwarze SR and Dowdy SF (2000) In vivo protein transduction: intracellular delivery of biologically active proteins, compounds and DNA. Trends Pharmacol. Sci. 21:45-48.

68. Remington: The Science and Practice of Pharmacy (Gennaro et al. eds., 20th ed. 2000).

1. A male contraceptive, comprising a peptide having an amino acid sequence corresponding to the second extracellular domain of a mammalian occludin, or an analog of said peptide, and a carrier, wherein said peptide or said analog thereof disrupts inter-Sertoli cell tight junctions in vivo.

2. The contraceptive of claim 1, further comprising a ligand that specifically Sertoli cells in the testis, wherein said ligand is linked to said peptide.

3. The contraceptive of claim 2, wherein said peptide and said ligand are linked by a peptide bond.

4. The contraceptive of claim 2, wherein said ligand comprises Follicle Stimulating Hormone (FSH).

5. The contraceptive of claim 1, which is in a form suitable for intratesticular injection.

6. The contraceptive of claim 1, which is in a form suitable for parenteral administration.

7. A method of contraception comprising administering to a mammalian male a composition comprising a peptide having an amino acid sequence corresponding to the second extracellular domain of a mammalian occludin, or an analog of said peptide, and a carrier, wherein said peptide disrupts inter-Sertoli cell tight junctions in vivo.

8. The method of claim 7, wherein said mammalian male is a human.

9. A method of producing a male contraceptive, comprising:
(a) preparing a peptide having an amino acid sequence corresponding to the second extracellular domain of a mammalian occludin, or an analog of said peptide, wherein said peptide disrupts inter-Sertoli cell tight junctions in vivo; and (b) combining said peptide with a carrier.

10. The method of claim 9, further comprising:
(1) preparing a ligand specifically targeting Sertoli cells in the testis; and
(2) linking said peptide and said ligand.

11. A peptide fragment of the second extracellular domain of a mammalian occludin, wherein said fragment may contain one or more substitutions, additions and/or deletions, and wherein said fragment disrupts inter-Sertoli cell tight junctions in vivo.

12. The peptide fragment of claim 11, wherein said fragment comprises the peptide sequence ALCNQFYTPAAT.

13. The peptide fragment of claim 11, wherein said fragment comprises the peptide sequence GSQIYTICSQFYTPGGTGLYVD.

14. A DNA molecule encoding a peptide having an amino acid sequence corresponding to the second extracellular domain of a mammalian occludin, or an analog, thereof, wherein said peptide or analog disrupts inter-Sertoli cell tight junctions in vivo.

15. The DNA molecule of claim 14, wherein a first portion thereof encodes said peptide and a second portion thereof encodes a ligand that selectively targets Sertoli cells in the testis, and wherein expression of said DNA molecule produces a fusion protein containing said peptide and said ligand.

16. The DNA molecule of claim 15, wherein said second portion encodes at least a portion of the binding domain of follicle stimulating hormone sufficient for said fusion protein to selectively target Sertoli cells in the testis.

17. The DNA molecule of claim 14, wherein said analog has an amino acid sequence corresponding to a fragment of the second extracellular domain of a mammalian occludin and which may differ from the native sequence of the fragment in terms of at least one amino acid substitution, addition or deletion.

18. A vector comprising the DNA molecule of claim 14.

19. A non-human host transformed with the DNA molecule of claim 14.

20. The non-human host of claim 19 which is E. coli.
